(19) United States
(12) Patent Application Publication
(10) Pub. No.: US 2015/0065545 A1
(43) Pub. Date: Mar. 5, 2015

(54) Transdermal Delivery of Anastrozole for Systemic Effect

(71) Applicants: Tsu-I Catherine Wang, Sugar Land, TX (US); Bruce Vincent Biundo, Houston, TX (US)

(72) Inventors: Tsu-I Catherine Wang, Sugar Land, TX (US); Bruce Vincent Biundo, Houston, TX (US)

(73) Assignee: Professional Compounding Centers of America, Houston, TX (US)

(21) Appl. No.: 14/011,552

(22) Filed: Aug. 27, 2013

(51) Int. Cl. A61K 31/4196

(52) U.S. Cl. CPC A61K 31/4196
USPC 514/383

(57) ABSTRACT

Formulations and methods for transdermal drug delivery compositions that include anastrozole are disclosed. Transdermal anastrozole compositions of the present disclosure may be indicated for treating testosterone deficiency. Disclosed transdermal anastrozole compositions may include permeation enhancers that may improve penetration of anastrozole in human skin. Permeation enhancers within transdermal anastrozole compositions may include oils from Amazon rainforest such as Pracaxi oil, Plukenetia volubilis seed oil, Inaja oil, and Patauá oil, which includes behenic and oleic fatty acids that may provide penetration power. Transdermal anastrozole may include organic solvents as transdermal penetration enhancers. Additionally, transdermal anastrozole compositions may include physiological lipids, phospholipids, and one or more butters rich in linoleic acid and linolenic acid that may also provide penetration power with restorative benefits to the skin.

### CROSS-REFERENCE TO RELATED APPLICATIONS

The present application is related to U.S. Ser. No. 13/xxx,xxx titled Testosterone Booster Transdermal Compositions, filed contemporaneously herewith.

### BACKGROUND

1. Field of the Disclosure

The present disclosure relates in general to transdermal delivery of pharmaceutical active ingredients, and more particularly to compositions and methods for anastrozole transdermal compositions.

2. Background Information

Anastrozole is an aromatase inhibitor (AI) that works by binding to the aromatase enzyme that converts testosterone into estrogen. Therefore anastrozole effectively inhibits or blocks conversion of testosterone into estrogen.

Generally speaking, the primary use of anastrozole for men is to suppress the production of estrogen, and treat testosterone deficiency. The suppression of estrogen, specifically the hormone estradiol, is often necessary for men who have hormone disorders. Elevated levels of the female hormone (estradiol) in men can be manifested in gynecomastia or growth of breasts in males and hypogonadism or the reduced function of the testes. Excess of estradiol can also contribute to increased risk of stroke, heart attack, chronic inflammation, prostate enlargement, and prostate cancer. Prescribing anastrozole for men in these situations has shown significant decrease in estradiol levels and, therefore, a decrease in symptoms and risks.

Transdermal drug delivery is receiving increased attention due to the ability of the administration regime to provide a controlled route for the release of an active pharmaceutical ingredient (API) to the systemic circulation. The delivery of drugs, such as anastrozole through the skin may provide many benefits; primarily, such a means of delivery is a comfortable, convenient, and noninvasive way of administering drugs. The variable rates of absorption and metabolism encountered in oral treatments are avoided, and other inherent inconveniences, such as gastrointestinal irritation, may be eliminated as well.

Numerous chemical agents have been studied for increasing the rate at which a drug penetrates through the skin. Chemical enhancers are compounds that are administered along with the drug (or in some cases the skin may be pretreated with a chemical enhancer) in order to increase the permeability of the stratum corneum, and therefore provide for enhanced penetration of the drug through the skin. Ideally, such chemical penetration enhancers or “permeation enhancers” are compounds that are usually innocuous and may serve merely to facilitate diffusion of the drug through the stratum corneum. The permeability of many APIs with diverse physicochemical characteristics may be improved using chemical enhancement means. However, there are skin irritation and sensitization problems associated with high levels of certain enhancers.

For the aforementioned reasons, there is a need for a noninvasive way to administer anastrozole, such as employing a transdermal composition of anastrozole that may include permeation enhancers, which may provide consistent delivery of anastrozole with reduced side effects.

### SUMMARY

A noninvasive way to administer anastrozole is disclosed. Compositions for transdermal anastrozole preparations that include anastrozole and permeation enhancers are disclosed. The disclosed transdermal anastrozole composition may allow the transdermal application of anastrozole and may reduce the risk of undesirable side effects in a patient. Transdermal anastrozole composition may include permeation enhancer compositions which may enhance the absorption of anastrozole. Methods for preparing transdermal anastrozole composition are also described.

In one embodiment, transdermal anastrozole compositions may include anastrozole as API and permeation enhancer compositions. Permeation enhancer compositions may include oils native to the Amazon Rainforest such as Pracaxi oil, Plukenetia volubilis seed oil, Inaja oil, and Patauá oil, among others. Amazon Rainforest oils may have essential fatty acids such as behenic acid, and oleic acid which may provide penetration power. Additionally, permeation enhancer compositions may include water, one or more skin lipids, one or more butters having linoleic acid and linolenic acid, and one or more phospholipids, among components. Physiological lipids, essential fatty acids, and phospholipids may provide penetration power with restorative benefits to the skin. In one aspect of the disclosure liposomes may be produced and may be present in the final permeation enhancer composition within transdermal anastrozole composition. Suitable additives, known to those skilled in the art, may be included in transdermal anastrozole composition.

In other embodiments, transdermal anastrozole compositions may include organic solvents as transdermal penetration enhancers, such as caprylic/capric triglycerides (medium chain triglycerides), ethyl alcohol, ethoxy diglycol, dimethyl sulfoxide (DMSO), glycerin, isopropyl myristate, isoproply palmitate, and propylene glycol, among others.

In one embodiment, amount of anastrozole included in transdermal anastrozole compositions may range from about 0.01% by weight to about 0.1% by weight, most suitable amount may be of about 0.01% by weight to about 0.1% by weight; and amount of permeation enhancer composition included in transdermal anastrozole compositions may range from about 5% by weight to about 50% by weight, most suitable amount may be of about 10% by weight to about 20% by weight.

According to other embodiments, amount of anastrozole included in transdermal anastrozole compositions may range from about 0.01% weight by volume to about 0.1% weight by volume, most suitable amount may be of about 0.01% weight by volume to about 0.1% weight by volume; and amount of permeation enhancer composition included in transdermal anastrozole compositions may range from about 5% weight by volume to about 50% weight by volume, most suitable amount may be of about 10% weight by volume to about 20% weight by volume.

In one embodiment, producing transdermal anastrozole compositions may be achieved by dissolving anastrozole in a penetration enhancer, such as caprylic/capric triglycerides (medium chain triglycerides), ethyl alcohol, ethyoxy diglycol, dimethyl sulfoxide (DMSO), glycerin, isopropyl myristate, isopropyl palmitate, and propylene glycol, among others, and the combination thereof. Subsequently, a gel base, or a cream base, such as PCCA Lipoderm®, may be added to bring to the final weight (or volume), with sufficient mixing.

Disclosed transdermal anastrozole may be applied on body surface as ointments, creams, gels, lotions, solutions, and pastes, among other suitable pharmaceutical preparations.

Transdermal anastrozole may be used for treating testosterone deficiency.

Transdermal anastrozole composition may be applied on body surface in a single dose that results in a pharmacologically effective blood concentration of testosterone over a suitable period of time. The dosage may be of from about 0.1 mg/day of anastrozole to about 1 mg/day of anastrozole. Most suitable dosage may be of from about 0.1 mg/day to about 1.0 mg/day of anastrozole. In one embodiment, transdermal anastrozole composition may be applied daily for an undetermined extended period of time. In other embodiments, transdermal anastrozole may be applied as prescribed by a doctor, according to the patient's need.

Numerous other aspects, features of the present disclosure may be made apparent from the following detailed description.

### DETAILED DESCRIPTION

The present disclosure is here described in detail. Other embodiments may be used and/or other changes may be made without departing from the spirit or scope of the present disclosure. The illustrative embodiments described in the detailed description are not meant to be limiting of the subject matter presented here.

### Definitions

As used here, the following terms have the following definitions:

“Treating” and “Treatment” refer to reduction in severity and/or frequency of symptoms, elimination of symptoms and/or underlying cause, prevention of the occurrence of symptoms and/or their underlying cause, and improvement or remediation of damage.

“Active Pharmaceutical Ingredient” or “API” refers to a chemical compound that induces a desired effect, and include agents that are therapeutically effective, prophylactically effective, or cosmeceutically effective.

“Therapeutically effective amount” refers to a nontoxic but sufficient amount of an active pharmaceutical ingredient to provide the desired therapeutic effect.

“Transdermal drug delivery” refers to administration of a drug to the skin surface of an individual so that the drug passes through the skin tissue and into the individual's blood stream, therefore providing a systemic effect.

“Body surface” refers to skin.

“Predetermined area of skin” refers to an area of skin through which a drug is delivered. It is intended for application on a defined area of intact unbroken living skin.

“Permeation enhancement” refers to an increase in the permeability of skin to the selected active pharmaceutical ingredient.

“Effective amount of a permeation enhancer” refers to a nontoxic, non-damaging but sufficient amount of the enhancer to provide the desired increase in skin permeability and, correspondingly, the desired depth of penetration, rate of administration, and amount of drug delivered.

“Vehicle” refers to a substance of no therapeutic value that is used to convey an active medicine for administration.

“Viscosity modulating agent” refers to a component of the composition which alters the viscosity of the overall resulting composition.

“Phospholipids” refers to fat-like organic compounds that resemble triglycerides, but have a fatty acid with a phosphate polar group.

“Liposomes” refers to artificially prepared vesicles made of lipid bilayer, and have concentric phospholipid bilayers.

“Butter” refers to a moisturizing product obtained of oils extracted from seeds and nuts. Butters are solid at room temperature, but melt on the skin.

“Lotion” refers to mixed phase or suspension of an API.

### Description

Embodiments of the present disclosure may be directed towards transdermal delivery of anastrozole. Compositions and methods for transdermal anastrozole compositions that may include permeation enhancers are described. The present disclosure includes anastrozole to treat testosterone deficiency.

Formulation

Transdermal anastrozole may include anastrozole as active pharmaceutical ingredient (API), permeation enhancers, suitable solvents, at least one viscosity modulating agent, and suitable additives.

In some embodiments, various additives, known to those skilled in the art, may be included in transdermal anastrozole composition to facilitate the preparation of suitable forms for patient's applications. For example additives may include humectants, pH adjusting agents, preservatives, emulsifiers, occlusive agents, opacifiers, antioxidants, fragrance, colorants, gelling agents, thickening agents, stabilizers, and surfactants, among others.

In one embodiment, transdermal anastrozole composition may include a viscosity modulating agent, such as a thickening agent or gelling agent. Concentrations of viscosity modulating agent may be determined by one skilled in the art, depending on the viscosity desired in order to obtain transdermal anastrozole composition that may be retained in the vicinity of the area of application for a brief period of time and allow increased uptake of APIs at the site.

In one embodiment, disclosed transdermal anastrozole composition may be a true solution. In other embodiments, viscosity of transdermal anastrozole compositions may be increased in order to obtain a lotion of transdermal anastrozole composition. Disclosed transdermal anastrozole may be applied on body surface as ointments, creams, gels, lotions, solutions, and pastes, among other suitable pharmaceutical preparations.

Anastrozole

Anastrozole is a non-steroidal aromatase inhibitor and is chemically described as 1,3-benzenediacetonitrile, a, a, a′, a′-tetramethyl-5-(1H-1,2,4-triazol-1-ylmethyl). The molecular formula of anastrozole is C₁₇H₁₉N₅.

Anastrozole acts by blocking the enzyme aromatase, subsequently limiting the amount of male hormones that are changed into estrogen by the aromatase enzyme, a process called aromatization.

Generally speaking, the primary use of anastrozole for men is to suppress the production of estrogen, the main female sex hormone. The suppression of estrogen, specifically the hormone estradiol, is often necessary for men who have hormone disorders. Elevated levels of the female hormone (estradiol) in men can be manifested in gynecomastia or growth of breasts in males and hypogonadism or the reduced function of the testes. Excess of estradiol can also contribute to increased risk of stroke, heart attack, chronic inflammation, prostate enlargement, and prostate cancer. Prescribing anastrozole for men in these situations has shown significant decrease of estradiol levels and, therefore, a decrease in symptoms and risks.

Furthermore, anastrozole has the ability to increase testosterone in the body. Some studies have shown that natural testosterone levels have increased as much as 60% after the use of anastrozole for about 7 days.

Additionally, the use of anastrozole may decrease fat mass, which can also be tied to estrogen levels.

In one embodiment, amount of anastrozole included in transdermal anastrozole compositions may range from about 0.01% by weight to about 0.1% by weight, most suitable amount may be of about 0.01% by weight to about 0.1% by weight.

According to other embodiments, amount of anastrozole included in transdermal anastrozole compositions may range from about 0.01% weight by volume to about 0.1% weight by volume, most suitable amount may be of about 0.01% weight by volume to about 0.1% weight by volume.

Permeation Enhancers

Permeation enhancer compositions may be added to transdermal anastrozole composition at a given percentage to give permeation power to transdermal anastrozole composition.

Permeation enhancer composition included in transdermal anastrozole composition may be a liquid or semi-liquid that includes phospholipids. Permeation enhancer compositions may include one or more naturally occurring substances, including one or more phospholipids, one or more oils rich in essential fatty acids (behenic acid, and oleic acid), one or more skin lipids, and one or more butters rich in linoleic acid and linolenic acid. The ingredients within permeation enhancer composition may act synergistically to increase the skin permeation to water and oil soluble products. When the permeation enhancer composition is prepared, liposomes may be formed from the fatty acids, including behenic acid and oleic acid that may be present in the one or more oils, and are stabilized by the phospholipids in permeation enhancer composition. More specifically, when permeation enhancer composition is added to water or a water-incorporating composition, liposomes may be formed.

Liposomes may be composed of naturally-derived phospholipids with mixed lipid chains or other surfactants. In some embodiments, the liposomes that are formed may be used to deliver APIs, transdermally to the skin's surface. Liposomes that are formed using embodiments of the present disclosure may be stabilized by the phospholipids.

Many types of phospholipids may be used in embodiments of the present disclosure. In one embodiment, the phospholipids used in permeation enhancer composition may include one or more of phosphatidylcholine, lysophosphotidylcholine, hydrogenated phospholipids, and unsaturated phospholipids. The polar end of the phospholipid molecule is hydrophilic, or soluble in water, and the other end or the fatty-acid end is hydrophobic, or soluble in fats. Phospholipids are ideal compounds for forming the biological membrane. There are two recognized classes of phospholipids, including phosphoglycerids, or those that have a glycerol backbone, and those phospholipids that include sphingosine. Examples of phosphoglycerids may include phosphatidic acid (phosphatidate) (PA), phosphatidylethanolamine (cephalin) (PE), phosphatidylcholine (lecithin) (PC), phosphatidylserine (PS), and phosphoinositides, which further include phosphatidylinositol (PI), phosphatidylinositol phosphate (PIP), phosphatidylinositol bisphosphate (PIP2), and phosphatidylinositol triphosphate (PIP3). Phospholipids that include sphingosine, also termed phosphosphingolipids, may include ceramide phosphorylcholine (sphingomyelin) (SPH), ceramide phosphorylethanolamine (sphingomyelin) (Cer-PE), and ceramide phosphorylglycereol. The most abundant types of phosphoglycerids are phosphatidylcholine (lecithin), phosphatidylethanolamine, phosphatidylserine, phosphatidylinositol, phosphatidylglycerol, and cardiolipin. Lysophospholipids (LPL) have a free alcohol in either the sn-1 or sn-2 positions. The prefix ‘lyso-’ comes from the fact that lysophospholipids were originally found to be hemolytic, but is now used to refer generally to phospholipids missing an acyl chain. LPLs may be the result of phospholipase A-type enzymatic activity on regular phospholipids, such as phosphatidylcholine or phosphatidic acid, although they can also be formed by the acylation of glycerophospholipids or the phosphorylation of monoacylglycerols.

Lysophosphatidylcholine (LPC) has been found to penetrate into the dermis faster than phosphatidylcholine, such that a small amount of LPC may penetrate the skin without damaging skin structure, and may be enzymatically degraded into several lipids.

Other components that may be included in permeation enhancer composition may be oils that are rich sources of essential fatty acids, behenic acid, and oleic acid. The supply of essential fatty acids and antioxidant molecules may restore the cutaneous permeability and the function of the skin barrier. The supply of essential fatty acids and antioxidant molecules may also contribute to the control of the imperceptible water loss and maintain moisture of the skin.

Behenic acid and oleic acid, when used by themselves, may be irritating when applied to the skin, which makes behenic acid and oleic acid difficult to use as permeation enhancers. While having an irritating effect on the skin, behenic acid and oleic acid may also be effective vehicles at delivering anastozole through the skin. In one embodiment, oil from a tree in Brazil has the highest natural sources of behenic acid and oleic acid. The tree is called Pentaclethara macroloba, or more commonly termed the Pracaxi tree. Pentaclethra macroloba seed oil, also called Pracaxi oil, is extracted from the tree, which includes high concentrations of behenic acid and oleic acid. Generally, Pracaxi oil may include about 20% of behenic acid and about 35% of oleic acid. In some cases, Pracaxi oil may include more than these percentages. As behenic acid and oleic acid are present in an oil, the effects of the acids may be less irritating on the skin, and as such makes Pracaxi oil a good choice for one of the ingredients of permeation enhancers.

In some embodiments, another oil that may be used, within permeation enhancer compositions, in combination with Pracaxi oil is Plukenetia volubilis seed oil, also known as Inca Inchi. Plukenetia volubilis seed oil is native to the Amazon Rainforest. Plukenetia volubilis seed oil extracted from the Plukenetia volubilis plant is one of the largest plant sources of the Omega family of fatty acids, including a high concentration of protein. Plukenetia volubilis seed oil is also rich in iodine and vitamin A and vitamin E. Plukenetia volubilis seed oil is a natural oil with an exceptional content in polyunsaturated fatty acids (greater than 90%) and tocopherols (1.5 to 2 g/kg). Plukenetia volubilis seed oil is a vegetable oil having both essential fatty acids in such a high amount, including 49% of alphalinolenic acid (omega-3) and 34% of linoleic acid (omega-6). While Plukenetia volubilis seed oil has a very high amount of fatty acids, Plukenetia volubilis seed oil also has high amounts of behenic acid (10-30%) and oleic acid (35-80%). When an oil such as Plukenetia volubilis seed oil and/or Pracaxi oil are used, behenic acid and oleic acid may work to enhance the restoration of cutaneous barrier organization and epidermal elasticity, in addition to contributing to the control of imperceptible water loss, thus, maintaining skin hydration. This is, at least in part, due to the high amounts of essential fatty acids in Plukenetia volubilis seed oil and Pracaxi oil. The link between skin permeation and hydration is clear. Increasing the permeability of the stratum corneum may be achieved by the increase of water content in the stratum corneum. Hydration by occlusion may cause a swelling of the corneocytes and subsequently may increase the skin permeation to anastrozole.

Still yet another oil that may be included in permeation enhancer compositions may be from a tree called Maximiliana maripa palm, or Inaja. Inaja oil has one of the highest sources of lauric acid (greater than 40%) and oleic acid (greater than 15%). Further, the highest concentration of fatty acids found in the Inaja may be found in kernel oil, as opposed to the pulp oil. Inaja is an indigenous Amazonian palm widespread in the state of Pará, growing around the Amazon River estuary. Oil from Inaja may be extracted from the fruits of the Inaja palm, which may include about 70% short-chain fatty acids, including lauric acid and myristic acid.

In further embodiments other oils such as Buriti, Patauá, Tucuma, Bacuri, Ucuuba, Muru-Muru, Andiroba, and Copaiba, among others, may be included in permeation enhancer composition within disclosed transdermal anastrozole composition.

Patauá oil may be extracted from the mesocarp of the patauá palm and generally appears as a greenish-yellow and transparent liquid, with little odor and taste, having the physical appearance and composition of fatty acids that are similar to olive oil (Olea europaea). Patauá oil includes high content of unsaturated fatty acids. Due to high content of oleic acid within patauá oil, patauá oil has moisturizing properties.

Andiroba oil may be extracted from the Carapa guianensis tree. Andiroba oil is rich in omega-3 fatty acid, which may be fast absorbed into the skin. The oil is also a rich source of essential fatty acids, including oleic, palmitic, myristic and linoleic acids, and includes non-fatty components, such as triterpenes, tannins, and alkaloids, which may be isolated as Andirobina and Carapina.

Copaíba balsam may be extracted from the bark of the Copaífera officinalis Jacq. tree where copaiba balsam accumulates in cavities within the tree trunk. The chemical composition of the oil-resin of Copaíba may include approximately 72 sesquiterpenes (hydrocarbons) and 28 diterpenes (carboxylic acids), and the oil may include 50% of each of these terpenes.

Ucuúba butter may be obtained from the seeds of the Virola sebifera Aubl tree. Ucuúba butter includes high concentrations of lauric, myristic and palmitic acid as well as vitamin C and A.

Bacuri (Platonis insignis) is an ornamental tree that may be found in the Amazonian forest, the seeds of which include high oil levels and high percentages of palmitic and oleic fatty acids.

Another component of permeation enhancer composition may be skin lipids. Examples of skin lipids that may be used in permeation enhancer composition may include ceramides and/or squalene. Ceramides are the major lipid constituent of lamellar sheets. Ceramides are a structurally heterogeneous and complex group of sphingolipids including derivatives of sphingosine bases in amide linkage with a variety of fatty acids. Differences in chain length, type, and extent of hydroxylation and saturation are responsible for the heterogeneity of the epidermal sphingolipids. Ceramides may play an important role in structuring and maintaining the water permeability barrier function of the skin. In conjunction with the other stratum corneum lipids, they form ordered structures. A structured semi-occlusive barrier that increases skin hydration may be a positive influence on the penetration of APIs.

Another skin lipid that may be used is squalene, which is a lipid fat in the skin. When used together with a ceramide and a phospholipid, such as phosphatidylcholine, the permeation enhancer composition may be mild such that permeation enhancer composition may be used on even sensitive skin. Squalene may also help to decrease water evaporation, thus speeding up skin permeation to APIs and decreasing irritation made by surfactants found in emulsions.

Yet another component of permeation enhancer composition may be butters rich in linoleic acid and linolenic acid. One example of linoleic acid and linolenic acid rich butters may be butyrospermum parkii butter, also known as shea butter. Other exemplary butters that may be used within permeation enhancer compositions may include cupuacu butter, buriti butter, passionfruit butter, mango butter, tucuma butter, palm butter, murumu butter, chamomile butter, cocoa butter, orange butter, lemon grass butter, avocado butter, tamanu butter, aloe butter, shea butter, monoi butter, pomegranate butter, almond butter, jojoba butter, red palm butter, acai butter, olive butter, matcha green tea butter, brazil nut butter, macadamia butter, kokum butter, mafura butter, coffee butter, tucuma butter, ucuuba butter, bacuri butter, and chamomile butter, among others.

In one embodiment, amount of permeation enhancer composition included in transdermal anastrozole compositions may range from about 5% by weight to about 50% by weight, most suitable amount may be of about 10% by weight to about 20% by weight.

In other embodiments, amount of permeation enhancer composition included in transdermal anastrozole compositions may range from about 5% weight by volume to about 50% weight by volume, most suitable amount may be of about 10% weight by volume to about 20% weight by volume.

In other embodiments, transdermal anastrozole compositions may include organic solvents as transdermal penetration enhancers, such as caprylic/capric triglycerides (medium chain triglycerides), ethyl alcohol, ethoxy diglycol, dimethyl sulfoxide (DMSO), glycerin, ispropryl myristate, isoproply palmitate, and propylene glycol, among others.

Methods of Elaboration

In one embodiment, producing transdermal anastrozole compositions may be achieved by dissolving anastrozole in a penetration enhancer, such as caprylic/capric triglycerides (medium chain triglycerides), ethyl alcohol, ethyoxy diglycol, dimethyl sulfoxide (DMSO), glycerin, isopropyl myristate, isopropyl palmitate, and propylene glycol, among others, and the combination thereof. A surfactant may be added to help solubilize anastrozole. Subsequently, a suitable surfactant, such as polysorbate 80 may be mixed in. Then, water may be added to bring to the final volume, and a gelling or thickening agent, such as hydroxypropyl cellulose, may be added in until homogeneity is achieved. In other embodiments, a gel base, or a cream base, such as PCCA Lipoderm ®, may be added to bring to the final weight (or volume), instead of bringing to the final weight (or volume) with water before adding the gelling agent. Transdermal anastrozole composition may be packaged in suitable containers.

Application

Disclosed transdermal anastrozole composition, when applied on a body surface, may deliver a therapeutically effective amount of anastrozole to the systemic circulation of the patient. In particular, transdermal anastrozole composition may be used to deliver a predetermined amount of anastrozole to achieve a predetermined bloodstream level of anastrozole.

Transdermal anastrozole composition may be used in treating a wide variety of conditions responsive to testosterone deficiency.

In one embodiment, transdermal anastrozole composition may be applied on body surface to restore testosterone level and maintain testicular function.

Some of the sequelae of adult testosterone deficiency include a wide variety of symptoms including loss of libido, erectile dysfunction, oligospermia or azoospermia, absence or regression of secondary sexual characteristics, progressive decrease in muscle mass, fatigue, depressed mood and increased risk of osteoporosis. Many of these disorders are generically referred to as male menopause.

In other embodiments, transdermal anastrozole composition may be applied on body surface of a patient that is in need of increased muscle mass. Transdermal anastrozole composition may also be administered to a patient that suffers from lipodystrophy and to a patient that is in need of increased lymphocyte levels. Transdermal anastrozole composition may also be administered to a patient in need of reduced triglyceride level or to a patient that suffers from benign prostate hypertrophy. Furthermore, transdermal anastrozole composition may be administered to a patient that suffers from prostate cancer or to a patient that suffers from a disorder related to male hypogonadism.

Different transdermal anastrozole formulations may be designed to provide higher or lower testosterone doses.

Transdermal anastrozole composition may be applied on body surface in a single dose that results in a pharmacologically effective blood concentration of testosterone over a suitable period of time. The dosage may be from about 0.1 mg/day of anastrozole to about 1 mg/day of anastrozole. Most suitable dosage may be of from about 0.1 mg/day to about 1.0 mg/day of anastrozole. In one embodiment, transdermal anastrozole composition may be applied daily for an undetermined extended period of time. In other embodiments, transdermal anastrozole may be applied as prescribed by a doctor, according to the patient's need.

Transdermal anastrozole may be applied on any suitable area of skin. Most suitable sites to apply transdermal anastrozole composition may be ventral forearm, upper arm, and chest. Disclosed transdermal anastrozole composition may be applied to those areas of skin which provide maximal systemic absorption due to increased cutaneous blood flow and heat.

In some embodiments, the amount of transdermal anastrozole composition administered is a defined, finite amount that provides a therapeutically effective amount (such as a single daily dose) of anastrozole.

The use of disclosed transdermal anastrozole composition may lead to increased flux of anastrozole, therefore, enabling a greater proportion of anastrozole in a dose to be delivered to the patient and using a smaller area of skin. A therapeutically effective amount of disclosed transdermal anastrozole composition may be applied to a minimal surface area, therefore minimizing the unpleasant side effects and patient inconvenience associated with applying large amounts of transdermal anastrozole composition to a large surface area.

In some embodiments, disclosed transdermal anastrozole composition may be applied manually with or without an applicator such as a dropper or pipette, an applicator such as a swab, brush, cloth, pad, sponge, or with any other applicator, such as a solid support including paper, cardboard or a laminate material, including material with flocked, glued or otherwise fixed fibers, among others. Alternatively, transdermal anastrozole composition may be applied as an aerosol or non-aerosol spray, from a pressurized or non-pressurized container. In further embodiments, transdermal anastrozole composition may be administered in metered doses, such as from a metered dose applicator or from an applicator including a single dose of transdermal anastrozole composition.

Transdermal anastrozole composition may become touch dry within about one to three minutes of application to body surface.

### EXAMPLES

Example #1 is an embodiment for formulation of transdermal anastrozole composition which includes the ingredients described in table 1.

TABLE 1
Example #1 transdermal anastrozole composition.

| INGREDIENTS | PERCENTAGE |
|---|---|
| Anastrozole | 0.01-0.1% |
| Penetration Enhancers | 5-50% |
| Base | q.s. to 100% |

Example #2 is an embodiment for formulation of transdermal anastrozole composition which includes PCCA Lipoderm® base as described in table 2.

TABLE 2
Example #2 transdermal anastrozole composition.

| INGREDIENTS | PERCENTAGE |
|---|---|
| Anastrozole | 0.01-0.1% |
| Penetration Enhancers | 5-50% |
| PCCA Lipoderm ® Base | q.s. to 100% |

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A pharmaceutical composition, comprising: anastrozole and at least one permeation enhancer.

2. The pharmaceutical composition of claim 1, wherein the anastrozole comprises about 0.01% to about 0.1% by weight of the pharmaceutical composition.

3. The pharmaceutical composition of claim 1, wherein the permeation enhancer comprises about 5% to about 50% by weight of the pharmaceutical composition.

4. The pharmaceutical composition of claim 1, wherein the permeation enhancer comprises about 10% to about 20% by weight of the pharmaceutical composition.

5. The pharmaceutical composition of claim 1, wherein the pharmaceutical composition is formulated as an ointment, cream, gel, lotion, solution, or paste.

6. A method of increasing hormone levels comprising: administering to a patient a pharmaceutical composition comprising anastrozole and at least one permeation enhancer.

7. The method according to claim 6, wherein the anastrozole is applied at about 0.1 mg/day to about 1 mg/day.

8. The method according to claim 6, wherein the administration of the pharmaceutical composition is to the skin.

9. The method according to claim 6, wherein the permeation enhancer is selected from the group comprising pracaxi oil, Plukenetia volubilis seed oil, Inaja oil, and Patauá oil, and combinations thereof.

10. The method according to claim 6, wherein the permeation enhancer is selected from the group comprising behenic acid, oleic acid, and combinations thereof.

11. The method according to claim 6, wherein the permeation enhancer further comprises water, one or more skin lipids, one or more butters having linoleic acid or linolenic acid, and one or more phospholipids, and combinations thereof.

12. The method according to claim 6, wherein the permeation enhancer comprises at least one medium chain triglyceride, ethyl alcohol, ethoxy diglycol, dimethyl sulfoxide, glycerin, ispropryl myristate, isoproply palmitate, propylene glycol, and combinations thereof.

13. The method according to claim 12, wherein the at least one medium chain triglyceride comprises at least one from the group consisting of caprylic triglyceride, capric triglyceride, and combinations thereof.

14. The method according to claim 6, wherein the anastrozole comprises about 0.01% to about 0.1% by weight of the pharmaceutical composition.

15. The method according to claim 6, wherein the permeation enhancer comprises about 5% to about 50% by weight of the pharmaceutical composition.

16. The method according to claim 6, wherein the permeation enhancer comprises about 10% to about 20% by weight of the pharmaceutical composition.

17. The method according to claim 6, wherein the pharmaceutical composition is formulated as an ointment, cream, gel, lotion, solution, or paste.
